(19) United States
(12) Patent Application Publication
(10) Pub. No.: US 2017/0361002 A1
(43) Pub. Date: Dec. 21, 2017

(54) INTEGRAL ARTIFICIAL HEART DEVICE

(71) Applicants: Gerard PLAS, Brissac (FR); Corentin PLAS, Brissac (FR); Gwen Sebastien PLAS, Penn Valley, CA (US)

(72) Inventors: Gerard PLAS, Brissac (FR); Corentin PLAS, Brissac (FR); Gwen Sebastien PLAS, Penn Valley, CA (US)

(21) Appl. No.: 15/541,489

(22) PCT Filed: Dec. 28, 2015
(86) PCT No.: PCT/FR2015/000243

§ 371 Date: Jul. 5, 2017

(30) Foreign Application Priority Data
Jan. 5, 2015 (FR) 15 00004
Dec. 24, 2015 (FR) 15 02697

(51) Int. Cl. A61M 1/10; A61M 1/12

(52) U.S. Cl. CPC A61M 1/1044; A61M 1/106; A61M 1/1098; A61M 1/122; A61M 1/1037

(57) ABSTRACT

Integral artificial heart device capable of storing venous blood in dynamic atria, without interrupting the continuous return of the blood. The device comprises a right ventricle (A1) and left ventricle (A2) pulsing simultaneously, and the reactive right atrium (C1) and left atrium (not illustrated) thereof, immersed in a pneumatic spec (D) having a variable vacuum D, which is driven by a solenoid (35), acting sequentially, by repulsion, on the permanent magnet discs (20, 21) included in the elastic ventricular membranes (18, 19), which beat simultaneously in the ventricular spaces (A1) and (A2), and, in the opposite direction, in pneumatic space (D) which houses elastic tubes acting as atria. The device simultaneously ejects systolic volumes, and accepts the proportion of continuously returning venous blood to store in the atria, during the systole, such that said continuous return is not interrupted by sequential systolic closure of the intake ports.

### FIELD OF THE INVENTION

The claimed device presented below is an integral artificial heart device meant to be implanted in the pericardial cavity as a replacement for a biological heart.

### RELATED ART

There exist several partial backup devices meant for a temporary use, usually to assist the left ventricle. They prelude the ambition to have an artificial heart functioning as a full replacement, which nowadays is called an integral artificial heart device.

Apart from claimed devices that have not been created and from attempts to create rotary pumps that are inadequate, all the suggested hearts that have been tested are characterized by the fact that the ventricular beats are alternate and successive instead of simultaneous. The reason for it seems to be that these suggestions do not have a dynamic atrium (also called an auricle depending on the terminology used, on the understanding that we will use the term atrium for the rest of this document). As a consequence, in this particular case, the duration of the diastole is necessarily equal to the duration of the beat, i.e. a duration that in its simultaneous natural setting is equal to the total of the duration of the systole and of the distinct duration of the diastole as we understand it, which is the duration of the filling up of the ventricular pump casings and not the duration of the permanent return of the initial systolic volume that corresponds to the duration of the beat, as one must understand it if one wants to take into account the intervention of the atria and of their functions as fully part of the venous return system of the bloodstream in a closed circuit, bearing in mind that this section of the venous circuit lies within the biological heart device.

In order to circumvent the contradiction that stems from this, until today developers have been forced to add up the two sequential durations that constitute the duration of a beat instead of the duration of the diastole. As a consequence each half-heart needs the total duration of a beat to fill its ventricle up, which implies the alternative character of the beats.

The current state of the art shows that while the creation of ventricular pumps is no real problem, there is still no reasonable solution for the creation of efficient atria.

### OBJECTS AND SUMMARY OF THE INVENTION

The biological heart is a device that is capable of combining harmoniously two circulatory systems that are consecutive and yet fundamentally different, contradictory and incompatible without the help of a combinatory and intermediate coordination and harmonization device in the process of receiving the returning venous blood during the systole, through temporary storage followed by the filling up of the ventricles in two phases during the diastole.

Indeed the systole, which is the convulsive phase when the blood is sent to the organs, is sequential and spasmodic in the arterial network, unlike the diastole, the phase when the ventricles fill up, that seemingly also operates in a sequential fashion but does so from a venous return flow that is permanent, constant and regular. This phenomenon is incompatible with the division of the global time of a beat between systole and diastole, taking into account the commonly agreed definition of the term “diastole”. Indeed, if the venous return blood flow cannot access the ventricles during the systole, the return flow of the closed circuit could not actually access the heart without the interventions of the atria that are adaptable to changing and constantly variable conditions. Without the intervention of this active, reactive, adaptable, synchronous temporary storage system, the permanent venous return flow would be interrupted in a sequential fashion. This would cause back fluid hammers that are destructive in the venous system, unless we admit that the venous return blood flow does not pause at the entrance of the atria but in front of the mitral orifice for the left half-heart and in front of the tricuspid orifice for the right half-heart. As a result of the above, the atria are indispensable.

As a consequence, apart from a general layout that by itself is original, the key element of the claimed device lies in the suggestion of a time control device as a replacement for the natural atria. This element has been missing in former suggestions of the state of the art. As such, the claimed device is a fully total substitute artificial heart comprising two (right and left) artificial ventricles with simultaneous pulses (sequential and spasmodic), coupled with their respective artificial atria (also right and left) that are reactive, synchronous and adaptable, and with the corresponding non-return valves that are necessary for the one-way flow structure. The atria and the ventricles are moved in a single motion by an original electromagnetic device. This device comprises no mechanical transmission element that can be subject to failure. It is controlled by an electronic device that controls the electromagnet and manages the flow variations depending on several parameters. Carbon dioxide and adrenaline sensors implanted in the right heart and two baroreceptors implanted in the ventricle and the atrium of the left heart measure these parameters.

### BRIEF DESCRIPTION OF FIGURES

The accompanying drawings illustrate the invention:

FIG. 1 represents a cavalier projection of the invented device at the end of the diastole.

FIG. 2 represents a cavalier projection of the invented device at the end of the systole.

FIG. 3 represents a front cross-sectional view of the invented device at the end of the diastole.

FIG. 4 represents a front cross-sectional view of the invented device at the end of the systole.

FIG. 5 represents a side cross-sectional view of the invented device at the end of the diastole.

FIG. 6 represents a simplified diagram of the cooling device of the electromagnet.

FIG. 7 represents the circular valves in a schematic fashion, the upper part representing an arterial valve and the lower part a venous valve.

FIG. 8 represents the construction method of the lamellar valves in a simplified way.

FIG. 9 represents an exploded view of the invented device at the end of the diastole.

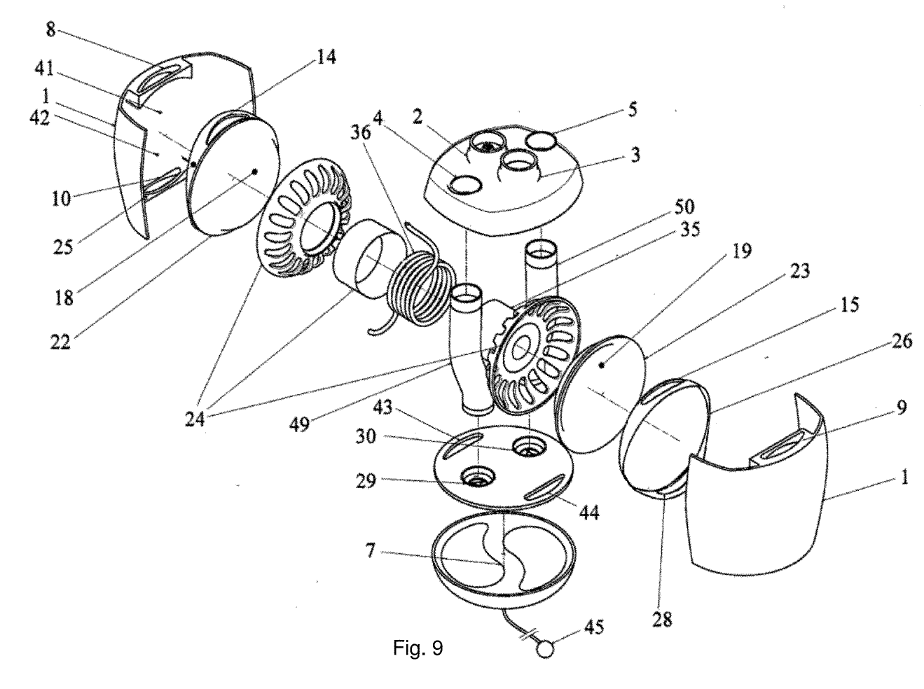

### DETAILED DESCRIPTION OF THE EMBODIMENTS

With reference to these drawings, the device comprises an ovoid rigid case (1) with external appendages that are indispensable for arterial connections (2, 3) and venous connections (4, 5) to the served biological vascular network. Inside the rigid case, various compartmentalization secondary elements (6, 7, 8, 9, 10, 11) can be found but they are fully part of the case, just like the above-mentioned communication and connection exterior appendages.

The case has a double-function. First, it separates the claimed artificial heart device from the rest of the organism in the pericardial cavity. Second, it functions as a skeleton to anchor the internal architecture of the concurring secondary devices.

In light of the above, the internal volumetric space of the rigid case (1) is structured so that four specific types of volumetric spaces are demarcated: three hydraulic ones (A, B, C) and one pneumatic one (D), which are characterized by their functions or by the functions of the concurring secondary devices inside them. Three of these types (A, B, C) are present as pairs and respectively identified as A1 and A2, B1 and B2, and C1 and C2. The fourth type, D, is unique, so that there are effectively seven internal volumetric spaces, each one being hermetic to the six others except for the necessary communication means that connect them. These connections are controlled by non-return valves, which ensure the one-way character of the conventional flows of the right and left half-hearts, in accordance with the biological model.

The hydraulic volumetric spaces A1 and A2 are diaphragm pumps that ensure the ventricular function. Their variable volumetric space is defined by the surface of the membranes (18, 19) and by their strokes, which are connected to a circular rigid support and presentation structure (22, 23). This structure is extended by a cylindrical skirt (25, 26) that ensures the impermeability of the compartment with the part of the case facing it, corresponding to the projection of the circular plan of the aforementioned membranes on the internal surface of the case (1).

The two membranes (18, 19) are positioned back-to-back and the rigid circular structures (22, 23) that keep them in their parallel planes are connected to each other by a skeleton structure (24). This skeleton structure results from a geometrical combination of two openwork cups and of a dividing cylinder, which circumscribes an electromagnet that is kept cool in a concentric way. In this way the skeleton structure sets and maintains the distance separating the membranes and the distance separating the circular structures (22, 23) of the membranes (18, 19) from the rigid case.

The cylindrical skirts (25, 26) that extend the circular structures of the membranes until they touch the rigid case (1) close the variable volumetric space in which the beat of the membranes ensures the pressurization of the venous blood.

The skirts (25, 26) are matched with oblong holes (14, 15, 27, 28). There are two per each ventricular volumetric space: one to receive the venous blood (27, 28) and the other one (14, 15) to send it to the arteries.

In their centers and within their walls, the membranes (18, 19) house permanent magnet discs (20, 21), enclosed in the elastomer of the membranes.

The permanent magnets housed in the membranes are positioned relative to each other so that the north pole of one (20) faces the south pole of the other (21).

The connecting structure (24) of the ventricular devices holds and maintains in its center a cooled fixed-core electromagnet (35). It is oriented and powered so that the north pole of the electromagnet be opposable to the north pole of one of the permanent magnets (20) and so that the south pole of the electromagnet be opposable to the south pole of the other permanent magnet (21), and vice versa, depending on the powering of the electromagnet.

The device comprising the permanent magnets (20, 21) housed in the ventricular membranes (18, 19), the connecting skeleton structure (24) and the electromagnet that sets all of it in motion represents the general animating device. It has two actions: a hydraulic one for the ventricular spaces (A1 and A2) and a pneumatic one for the volumetric space (D) that is described below.

The general animating device is tangent to the dividing partition (6) via the circumference of the structures (22, 23) and of the cups of the connecting structure (24). The plane of the dividing partition is parallel to the axis that goes through the centers of the cups and of the general electromagnetic animating device. The dividing partition (6) isolates in the rigid case a second type of hydraulic volumetric space (B), which enables transit and return. This volumetric space is divided in two (B1 and B2) by the subdividing partition (7) that is perpendicular to it. The B1 and B2 spaces receive and return the venous blood coming from the atria C1 and C2 through the circular slots (29, 30) in the dividing partition (6) and send them to the receiving oblong slots (27, 28) of the right (A1) and left (A2) ventricles thanks to the exit oblong slots (43, 44) of the spaces B1 and B2. These exit oblong slots are located in the dividing partition (6) and face the oblong slots (27, 28), with which they communicate through folded back double-wall, oblong-cut canals (10 and 11).

The transit spaces B1 and B2 are connected to the atria C1 and C2 and communicate with them through two oblong slots (27, 28). The latter are equipped with non-return one-way valves (31, 32).

Secondarily, the cylindrical skirts (25 and 26) connected to the structures and the cups are tangent to the plane of the dividing partition (6), so that from the oblong exit slot of the dividing partition (6) to the corresponding oblong slots (25, 26) of the cylindrical skirts, it is necessary to confine these two slots in folded back double-wall canals (10, 11) connected to the rigid case, the dividing partition (6) and the skirt. This makes it possible for these appendages (10, 11), which are accessory to the rigid case (1), to ensure the impermeability of the communicating spaces (B1 and A1, B2 and A2) from all the other volumetric spaces and to lead the venous flows of the atrium spaces (C1, C2) through the transit spaces (B1, B2) to the slots (25, 26) of the ventricular skirts.

The hydraulic spaces C1 and C2 comprise two elastic pipes (49, 50) that are irregular, identical, symmetrical and with a variable volume. They link the venous connection appendages (4, 5) of the rigid case to the circular slots (29, 30) of the dividing partition (6). C1 and C2 have a variable volume and are preformed so that they can envelop the connecting skeleton structure (24) while avoiding that the pipes (49, 50) come into a surface contact with the rigid case or with the connecting skeleton structure (24) of the general electromagnetic animating device during its maximal dilatation. This dilatation must be able to hold half of the maximal systolic volume added to the dead volume determined during the relaxation phase. The latter must be made as small as possible in the design.

Adding the spaces A1 and A2, B1 and B2 and C1 and C2 and subtracting the internal total volume of the rigid case (1) determines a unique volumetric space (D). This volumetric space D houses the structure (24) and the electromagnet (35), but also the immersed atria C1 and C2, so that the volumetric space D is filled with a gas, function of its density and elasticity coefficient. This gas works as an elastic pneumatic transmission and as a diffuse pneumatic spring. As a consequence, by design and in virtue of the common beat of the ventricular membranes in the volumetric space D, this space D also has variable volume and depression, and the variation of the pneumatic depression in space D affects the walls of the pipes (49, 50) of the atria (C1, C2). It affects them so as to modify their resistance to the internal constant returning venous blood pressure that occurs inside the walls of the pipes (49, 50) of the atria C1 and C2.

It is this peculiarity, i.e. filling up the space D with a gas and its variable depression pneumatic behavior, from which the claimed artificial heart device mainly derives its originality. The gas, due to its elasticity, absorbs a part of the depressor torque that is led by the outward moves of the ventricular membranes. As a consequence only a part of the depressor torque manifests itself as an effective, active and relative depression torque on the walls of the pipes (49, 50) of the atria C1 and C2. These atria are thus conditioned to receive and store only the part of the venous flow that is proportional to the shutter time of the systole, so that if following this proportional and relative effect the atria C1 and C2 open to the returning venous blood flow while waiting for the active diastole phase, they will only receive the desirable volume and will not be able to tap the venous system by playing the inopportune role of a suction pump, which would have the catastrophic effect of depressing the veins of the return blood system.

Secondarily, the management of the arterial bloods and their conveyance towards the exterior connection appendages (2, 3) via the volumetric space D require two interior double-wall canals (8, 9) folded back from the rigid case, so that this secondary structure house and lead the blood towards the connectors (2, 3) of the natural inlet channel while ensuring the impermeability of the volumetric spaces A1 and A2 from the volumetric space D.

As mentioned, this volumetric space D houses two elastic pipes (49, 50), which are identical and of an irregular shape adapted to the maximal exploitation of the available extension volume in the spaces C1 and C2 that are immersed in the pneumatic space D.

The variable volumetric space of the spaces C1 and C2 represents the fourth type of volumetric space and is characterized by the fact that C1 and C2 receive the venous blood while waiting for the ventricular diastolic admission and store them via a controlled expansion that is led and synchronized by the systole. Thus C1 and C2 take on the role of atria.

The pipes (49, 50) are connected to the dividing partition (6) and open onto the spaces B1 and B2 via the aforementioned circular slots (29, 30). They are also connected to the vena cava and to the pulmonary veins via the connection appendages (4, 5) leading to the natural venous inlet channels, with the understanding that their representations as figures are purely schematic and do not portray their shape or practical structure within the context of an anatomical realization.

Secondarily, it is necessary to design the shape and thickness characteristics of the elastic pipes (49, 50) so that the dead volume of the pipes added to half of the maximal systolic volume when both are at their maximal expansion within the limits of the available rigid parts of the space D be acquired at the end of the systole, while avoiding that they prevent the expansion of the atria C1 and C2 and avoiding that the pipes (49, 50) come into a surface contact with their environment. In order to save space and for a proper use of the available expansion space, the dead volumetric space of the pipes must be as small as possible.

Secondarily, the general organization requires the insertion of communications means, i.e. non-return valves between the atria C1 and C2 and the ventricles A1 and A2 as well as between the ventricle sending slots and the connection appendages leading to the natural arteries.

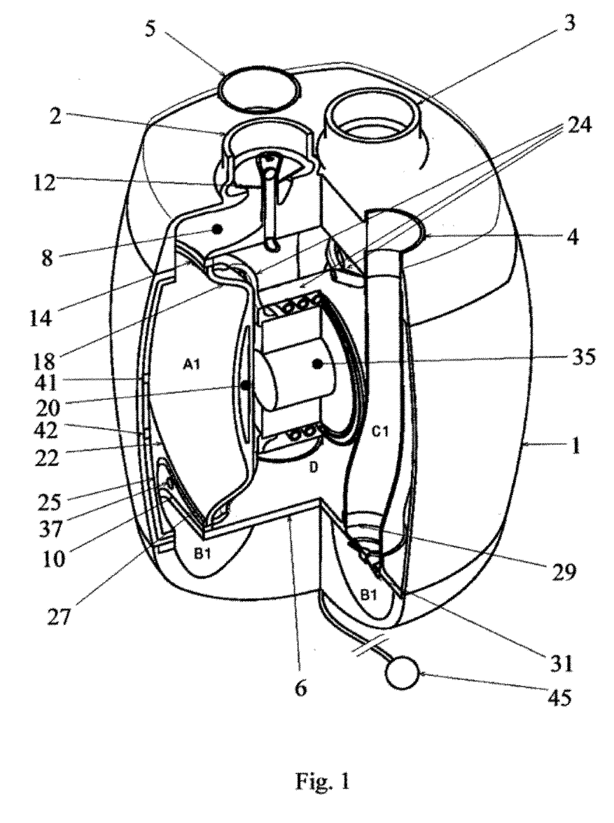

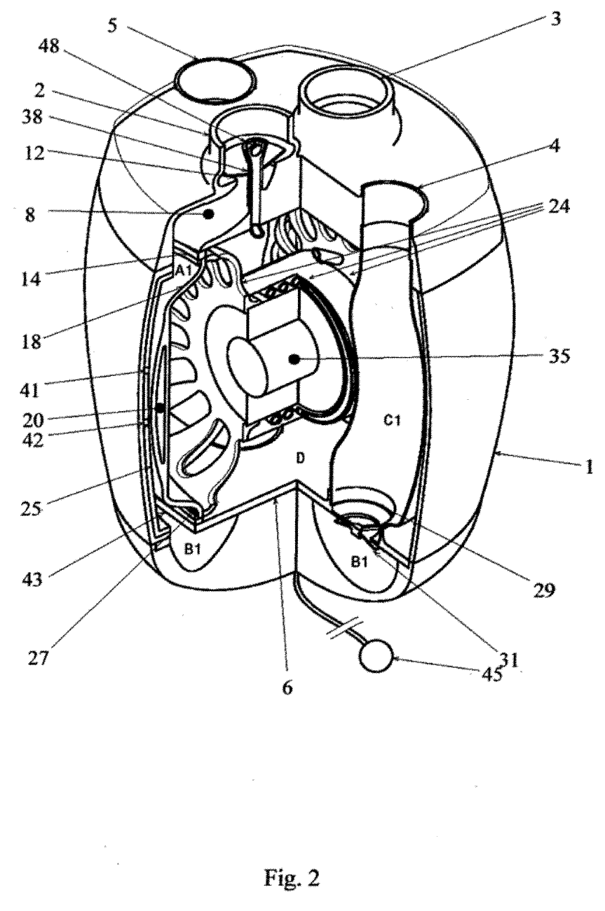

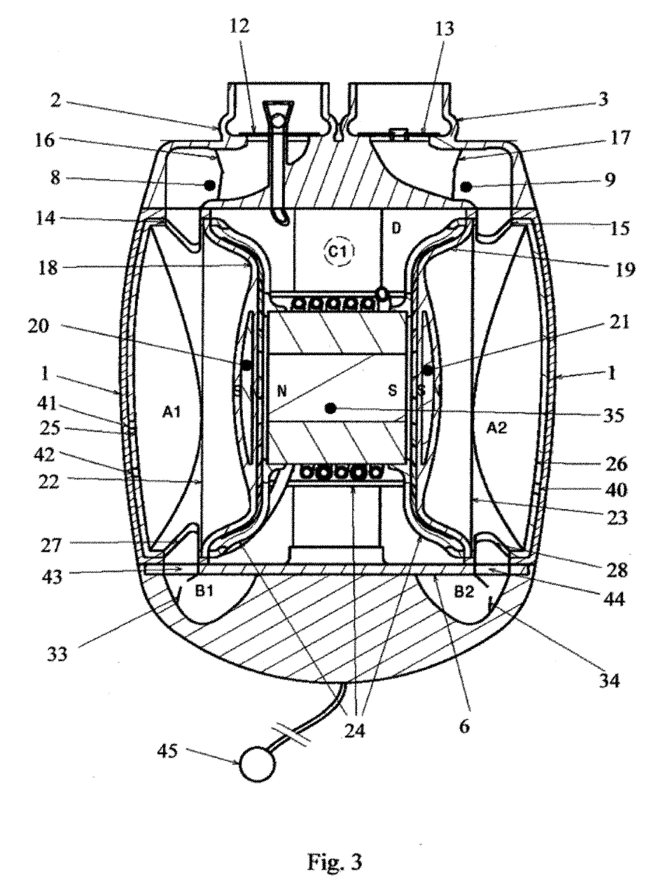

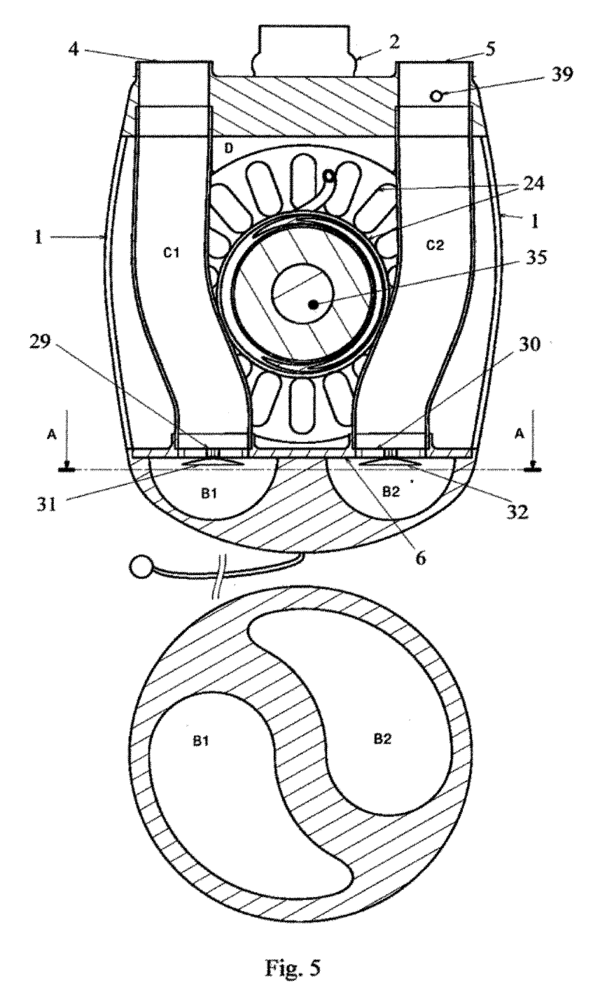

There are two distinct types of valves, as can be seen in the FIGS. 3, 4, 5, 7 and 8.

The first valve type is a classical type. It consists of a circular elastic veil made of elastomer (54), in the center of which a cylindrical fastening appendage rises and is housed by a pipe collar that is connected to a peripheral concentric ring via three hydrodynamic section branches. The collar, the ring and the three connecting branches form the fastening and presentation frame (46) of the elastic veil (54), so that the veil rises and falls back on the edge of the collar of its presentation frame that functions as a circular seat (46). This frame is installed in the circular slots (29, 30) of the dividing partition (6) in order to isolate the atria C1 and C2 from the transit spaces B1 and B2 and in the connecting appendages (2, 3) of the natural arteries for the systolic ejection.

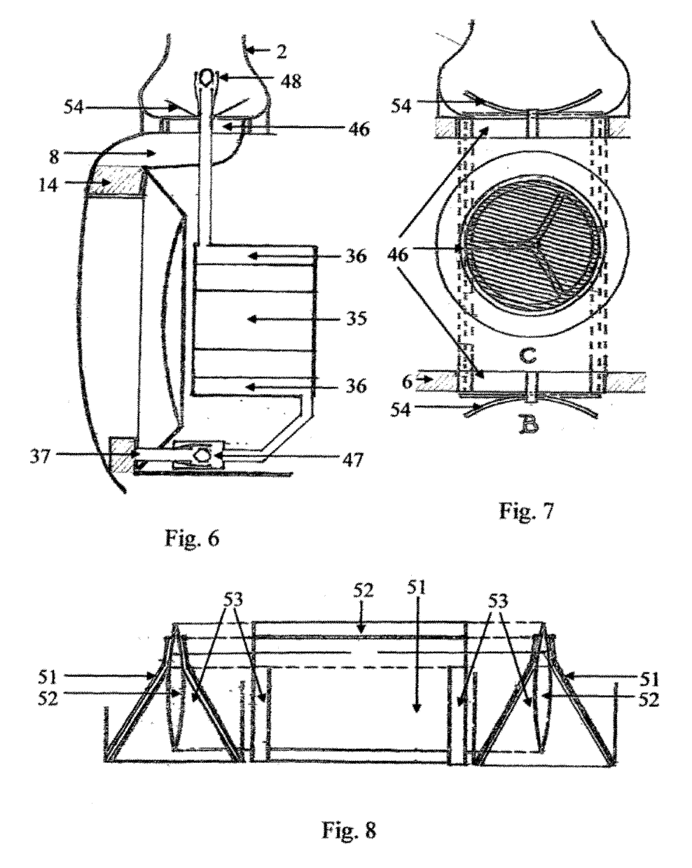

The second valve type (16, 17, 33, 34), which can be inferred from the oblong aspect of the skirt slots of the spaces A1 and A2, consists of two separate elastic blades (51) made of elastomer, as seen in FIG. 8. The blades close up in a single motion on a rigid, median support blade (52) that is fastened and presented by two triangles (53). The support blade and the triangles form a trestle, a structure that allows a great laxity of the elastic blades.

In accordance with the FIGS. 3, 4 and 8, these lamellar valves are installed in front of the oblong receiving slots (27, 28) for the venous blood in the spaces B1 and B2, and in front of the arterial connection appendages (2, 3) and of their circular valves (12, 13) in the arterial outlet canals (8, 9) for the arterial systolic blood.

The surface of the section of the aortic trunk for an individual of medium height and normal weight is commonly admitted as having a value of five square centimeters.

By virtue of this contingency, it is desirable that all the communication slots offer a section surface that is equal or slightly superior to five square centimeters.

The suggested and claimed organization keeps the possibility to fulfill this requirement.

Furthermore, it is necessary to install baroreceptors (39, 40) on the rigid parts of the ventricular cavity A2 and of the atrium C2 of the left half-heart and to connect them to an electronic control device (45) that control the electromagnet (35). This device serves first as a primary switch to open only the electrical circuit linked to the electromagnet (35). Second, since it is also linked to sensors for carbon dioxide (41) and adrenaline (42) that are located on the internal side of the case wall of the right half-heart ventricle A1, it regulates the electrical impulses that are sent to the electromagnet and whose length and intensity depend on the levels of carbon dioxide and adrenaline measured by the aforementioned sensors.

The electronic control device (45) must be detached on the exterior part of the claimed device and must be brought under the skin in order to facilitate fine-tuning, programming and adjustment procedures without surgical operations.

Furthermore, the functioning of the electromagnet (35) entails an inopportune Joule effect that requires organizing an ancillary cooling means in order to disperse the calories produced by the electromagnet.

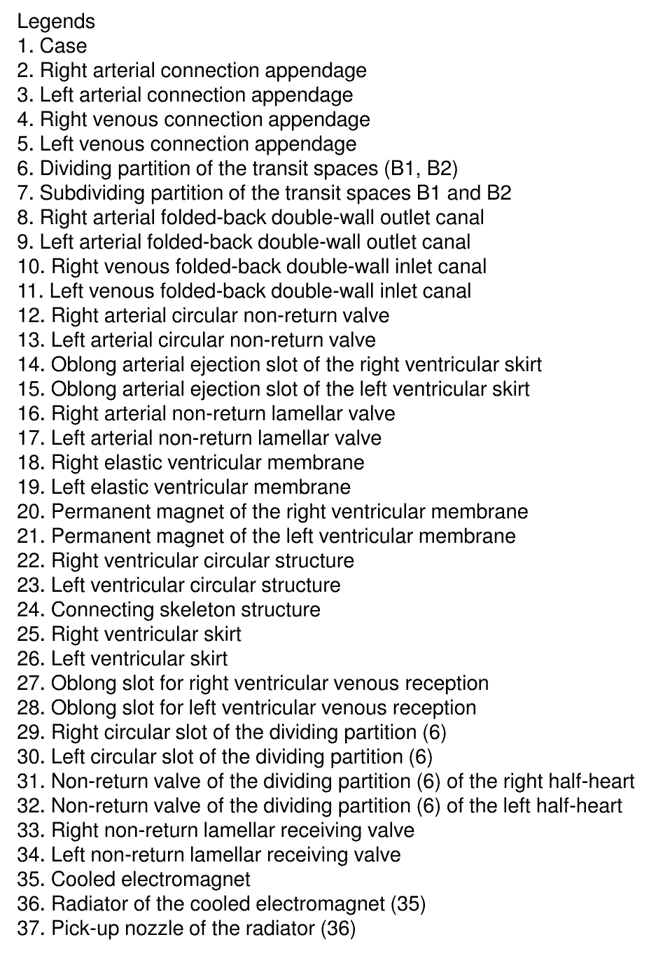

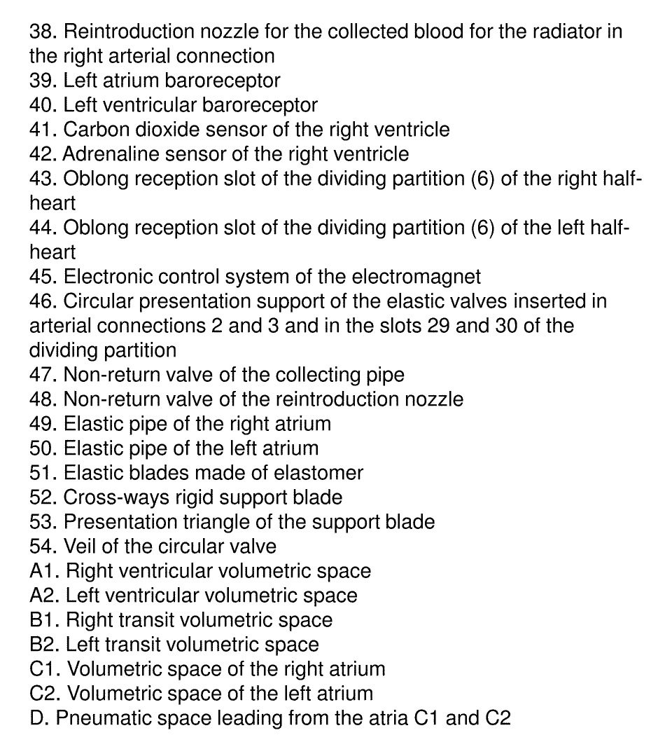

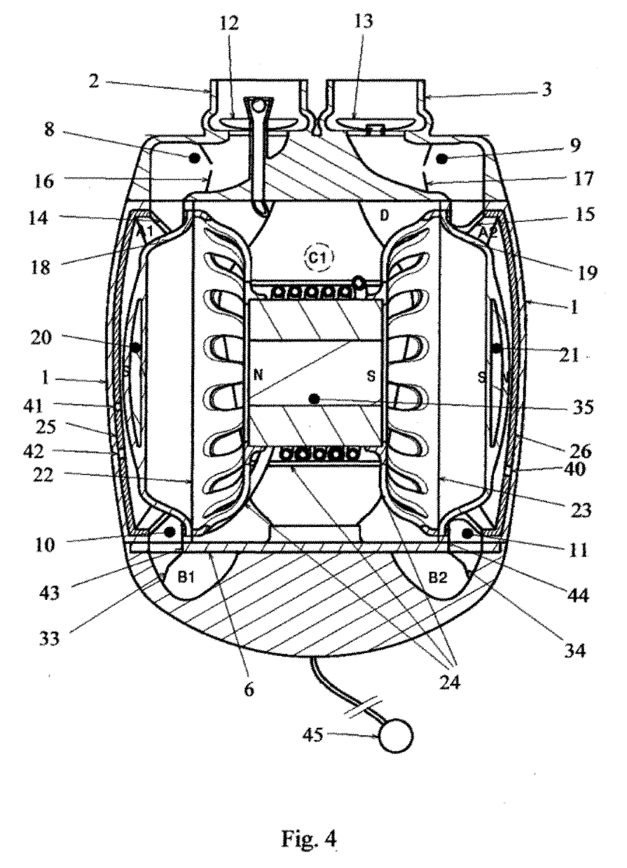

To this purpose, a small percentage of the venous blood must be diverted in the right half-heart via a hydraulic pick-up nozzle (37) equipped with a non-return valve and leading to the inlet canal of the right ventricle (10). The diverted blood is sent to a peripheral radiator (36) of the electromagnet (35). Afterwards it is sent again to be reintroduced in the connecting appendage (2) of the right half-heart downstream of its circular valve (12) via a reintroduction piping (38) equipped with a non-return valve and installed in a Venturi, as can be seen on the FIGS. 1, 2, 3, 4 and 6.

Functioning:

After connecting to the natural inlet channels and purging the claimed device entirely:

When resting, the ventricles are full and ready for the systole. At this stage the electromagnet (35) is not being powered. The functioning can then begin.

To this purpose, since the baroreceptors (39, 40) are balanced as a couple in the left half-heart, the electronic control device (45) can send and regulate an electrical impulse that is adapted to the current length and intensity needs determined by the levels of carbon dioxide and adrenaline in order to put in motion the electromagnet (35).

Systole:

The electrical impulse sent to the electromagnet solenoid (35) causes the magnetization and polarization of the soft iron core.

The permanent magnet discs (20, 21), housed in the membranes (18, 19), which were previously attracted to the center by the inert soft iron at the end of the diastole are opposed to the electromagnet poles (as mentioned in the description), so that the permanent magnets are pushed outwards.

The membranes (8, 9) are then thrown towards the rigid case in the ventricular spaces A1 and A2 for the systolic spasmodic ejection.

The variable stroke of the membranes determines the variable volume of the systolic ejection.

This movement of the membranes, which entails a reduction of the ventricular volume and thus a pressurization of the blood in the ventricular spaces A1 and A2 for the ejection of the systolic volume to the arteries, also entails at the same time a receding movement in the space D, so that this movement of the membranes causing a reduction of the volumetric space of A1 and A2 causes an equivalent compensatory increase of the volume of the pneumatic space D.

This volume increase increases the pneumatic depression of the volumetric space D. This depression has an impact on the walls of the atria C1 and C2, weakening them and thus dilating them. This dilatation, combined with the constant pressure of the return blood flow, increases the volume of the atria, which then receive the venous blood flow in order to store it in a temporary and synchronous fashion, with no interruption of the return blood flow.

As soon as the systole is over, as established by the electronic control device (45), the electrical impulse is interrupted.

Diastole:

The solenoid is not powered anymore. The electromagnet (35) is deactivated.

As the fixed soft iron core is not polarized anymore, it attracts again the permanent magnets (20, 21) of the membranes (18, 19), which also attract each other since their opposed poles are different (as mentioned in the description).

This passive magnetic recall, combined with the active pneumatic depression of the volumetric space D playing the role of a diffuse recall spring and with the relaxation of the distension of the pipes (49, 50) of the atria C1 and C2 that also has a recall spring effect, leads to the receding of the ventricular membranes (18, 19) and thus to a depression in the spaces A1 and A2 while the pressure increases in the atria C1 and C2.

This combination of forces first leads to the purge of the stored blood towards the ventricular spaces A1 and A2, which are in a state of depression due to the receding of the ventricular membranes (18, 19), via the transit spaces B1 and B2, so that while the pressurized purge of the spaces C1 and C2 is sucked up by the ventricular depression, the excessive pressure of the atria goes unnoticed by the return venous blood system downstream of atria C1 and C2 and does not modify the pressure of the upstream return flow in the venous system.

At the end of this initial phase of the diastole, the state of depression of the pneumatic space D is back to normal and the returning blood in a continuous flow finishes filling up the ventricular spaces A1 and A2 via the spaces B1 and B2, until the membranes be returned to the maximal receding position.

At this stage the pressures observed in the ventricle A2 of the left half-heart and in the atrium C2 of the left half-heart reach equilibrium.

The baroreceptor couple (39, 40) notices the barometric equilibrium and allows the next electrical impulse to be sent by the electronic control system (45), which regulates its length and intensity depending on the other parameters (carbon dioxide and adrenaline). The cycle is complete.

The efforts of the left and right half-hearts occur in a symmetrical and opposed fashion on both sides of the device's center of gravity. Theoretically the whole device is stable by itself and is thus externally inert inside the pericardial cavity inside the rib cage. This is worth noting considering the potential inopportune inflammatory reactions, which logic commands us to avoid as much as possible.

Furthermore, we must specify that for a practical realization and an effective implementation of the claimed artificial heart device, it is imperative that all the used material be blood-compatible for the parts that come into contact with the blood of the receiver and be at least biocompatible for the parts that come into contact with the rest of the organism.

1-12. (canceled)

13. An integral artificial heart device implantable in the pericardial cavity, comprising a right ventricle and a left ventricle, said ventricles exhibiting simultaneous pulses, a right atria and a left atria immersed in a variable pneumatic space, the ventricles and atria driven by an electromagnet acting sequentially by repulsion on permanent magnets housed in elastic ventricular membranes beating simultaneously in ventricular spaces, and, in an opposite direction, in the pneumatic space,
the pneumatic space housing atria consisting of elastic pipes, said device making it possible to simultaneously eject systolic volumes and accept proportion of continuously returning venous blood to store in the atria during the systole, such that said continuous return is not interrupted by a sequential systolic closure of intake ports.

14. The device according to claim 13, that comprises a case divided into six hydraulic volumetric spaces and the pneumatic space, a crosswise structure being integrally installed inside said case and consisting of ventricular skirts, the structures and an intermediary connecting structure, which holds the ventricular membranes and the permanent magnets.

15. The device according to claim 14, that comprises, between said membranes, a cooled electromagnet which, by interacting with the permanent magnets, forms a general electromagnetic stimulation device.

16. The device according to claim 15, wherein said ventricular membranes, rigid portions of the case and the ventricular skirts delimit, at a center of the case, the pneumatic space.

17. The device according to claim 16, wherein the membranes, the skirts and the case form ventricular hydraulic volumetric spaces, the device comprising non-return valves between the atria and the ventricles, and non-return valves between the ventricles and arterial outlets.

18. The device according to claim 13, that comprises sensors for carbon dioxide and adrenaline, and baroreceptors, connected to an interactive electronic control device positioned externally to the device, and a cooling radiator for the electromagnet.

19. The device according to claim 13, that comprises an ovoid rigid case, which acts as a skeleton for the internal anchoring of concurring devices, the case comprising, on the outside, communication appendages for connecting to natural inlet channels, two for arteries fitted with elastic non-return valves and at least two for veins, wherein the rigid case comprises, inside said case, a combination of concurring partitions,
an isolating partition isolating a specific hydraulic returning and transit spaces, said partition comprising two circular intake ports, fitted with non-return valves, and two oblong exit ports, and
a subdividing partition in which a plane of symmetry is perpendicular to the isolating partition, such that, in contrast to said appendages, said combination of partitions isolates two symmetrical hydraulic transit and returning pits.

20. The device according to claim 19, wherein the rigid case comprises at least two folded-back double-wall canals, including one canal for a right half-heart, and another canal for a left half-heart, such that the two canals channel the venous blood flows from the hydraulic transit spaces via the oblong ports of the partition towards intake ports of the ventricular hydraulic spaces, said canals being fitted with lamellar valves.

21. The device according to claim 20, wherein the rigid case also comprises an additional canal for the right half-heart and another additional canal for the left half-heart, such that the additional canals channel the blood flows from exit ports of the ventricular hydraulic spaces towards arterial outlet channels, these additional canals being fitted with non-return lamellar valves.

22. The device according to claim 19, wherein the atria consist of irregular, identical, symmetrical enveloping preformed elastic pipes linking connecting appendages of a case to transit and returning spaces via a ports open in the isolating partition, the pipes being immersed in a variable depression atmosphere of the pneumatic space, such that the variations in the pneumatic depression in the pneumatic space have an effect on elasticity of walls of the pipes that enclose the atrium spaces that induces the variation in the elasticity of the pipes and their subsequent expansion under the effect of the internal pressure of the venous blood that fills the pipes, allowing the pipes to receive the returning venous blood for temporary storage during the systole.

23. The device according to claim 13, that comprises a crosswise structure and a case, said structure comprising, from anchor points of the inner wall of the case, two ventricular skirts, connected to supporting structures, said structures being connected back-to-back to each other via a connecting structure, such that said connecting structure maintains and presents the membranes and permanent magnet housed in said membranes and an intermediate cooled electromagnet.

24. The device according to claim 23, wherein the ventricular membranes consist of a preformed elastomer veil incorporating a permanent magnet.

25. The device according to claim 24, wherein the membranes are held at edges by the support structures, extended by skirts, and anchored on the wall of the case, such as to enclose the ventricular spaces, the membranes beating in said ventricular spaces which comprise, open in the skirts, oblong intake ports served by the venous intake canals, as well as oblong arterial exit ports serving the arterial outlet canals.

26. The device according to claim 23, wherein the crosswise structure holds and presents the ventricular membranes and permanent magnets housed by said ventricular membranes, such that the north pole of one permanent magnet faces the south pole of the other permanent magnet, and the connecting structure holds a cooled electromagnet between the two housed permanent magnets, such that a north pole of the electromagnet is facing the inner north pole of one of the magnets while a south pole of the electromagnet is facing the inner south pole of the other permanent magnet, this device inducing, by magnetic repulsion, the simultaneous beating by the membranes, connected to the housed magnets, in the ventricular spaces and in the pneumatic space, such that during the systole said beat simultaneously constricts the hydraulic ventricular spaces and depresses the pneumatic space, and vice-versa during the diastole.

27. The device according to claim 26, wherein the variable pneumatic space, which separates the ventricular membranes and houses a cooled electromagnet, is filled with a gas which, as a function of a density of the gas and of an elasticity coefficient of the gas, is in a state of slight depression during its relaxation at the end of the diastole, such that the membranes simultaneously beat in the ventricular spaces and in the pneumatic space in the opposite direction, and consequently a pneumatic atmosphere of said pneumatic space has a variable depression, driven by the action of said membranes due to the elasticity of the filling gas.

28. The device according to claim 26, that comprises a radiator peripheral to the electromagnet, through which runs a small proportion of the blood diverted from a ventricular space during the systole via a nozzle fitted with a non-return valve and installed in a rigid portion of a skirt or in an inlet canal, the nozzle being connected to the radiator via an inlet pipe fitted with a non-return valve, the diverted blood volume being directed to an arterial outlet via a canal that ends with a nozzle fitted with a non-return valve and installed to form a Venturi tube in said arterial outlet.

29. The device according to claim 26, that comprises a primary electronic device for initially switching on the electrical pulse, one baroreceptor installed in a ventricular space and one baroreceptor installed in an atrium space, such that, coupled with an electronic control device of the electromagnet, the primary electronic device measures the balance of the immediate pressure and authorizes the delivery of the electrical pulse that is necessary for the electromagnet at the beginning of the systole.

30. The device according to claim 26, that comprises an electronic device for modulating and interrupting the electrical pulse of the electromagnet, and an electronic control device positioned externally to the integral artificial heart device and connected to sensors for carbon dioxide and adrenaline installed in a ventricular space, such that, based on the measured levels of carbon dioxide and adrenaline, the electronic control device modulates an intensity and a duration of electrical pulses for the systole.
